(19) United States
(12) Patent Application Publication
(10) Pub. No.: US 2022/0246297 A1
(43) Pub. Date: Aug. 4, 2022

(54) Causal Recommender Engine for Chronic Disease Management

(71) Applicant: Anthem, Inc., Indianapolis, IN (US)

(72) Inventors: Theodore Goldstein, Los Altos, CA (US); Bobby Samuel, Redwood City, CA (US); Beau Norgeot, Los Gatos, CA (US); Chinmay Belthangady, San Jose, CA (US); Stefanos Giampanis, Redwood City, CA (US); William Stedden, Fort Bragg, CA (US)

(21) Appl. No.: 17/321,456

(22) Filed: May 16, 2021

Related U.S. Application Data
(60) Provisional application No. 63/144,357, filed on Feb. 1, 2021.

(51) Int. Cl. G16H 50/20; G16H 50/30; G06K 9/62

(52) U.S. Cl. CPC G16H 50/20; G06K 9/6223; G16H 50/30

(57) ABSTRACT

The present application describes a system and method for implementing a causal recommender for personalized disease treatment selection using machine learning. The method includes obtaining health trajectories for patients. Each health trajectory includes sub-trajectories. Each sub-trajectory includes a treatment event and ends at a respective index event. The method further includes stratifying the sub-trajectories for each patient to form stratified patient segments. Each segment corresponds to a separate and distinct health condition and includes the sub-trajectories for patients that have the health condition. For each segment, the method includes performing pairwise causal inference analysis on one or more treatments to estimate average treatment effect (ATE) values, and performing network meta-analysis on the ATE values, thereby ranking the one or more treatments. The method also includes reranking the one or more treatments after excluding unsafe treatments, and outputting treatment options based on ranked treatments for the segments.

### TECHNICAL FIELD

The disclosed implementations relate generally to healthcare applications and more specifically to a method, system, and device for machine learning derived personalized disease treatment recommendations.

### BACKGROUND

Healthcare providers encounter situations in which there are multiple guideline-endorsed treatment options available but no clear best choice for an individual patient. In such cases, it would be helpful to have an up-to-date efficacy comparison across all treatment options to guide decisions. Given the nature of bias and confounding in medicine, it is a requirement that these comparisons be conducted in a causal framework to understand the effect of the treatment choice itself. However, it is arduous to do massively multi-comparator randomized control trials (RCTs) of already approved treatments due to scale, cost, and time. To date, observational causal framework methodologies have been practically limited to working with only a few simultaneous trial arms.

### SUMMARY

Accordingly, there is a need for an automated causal recommender system (e.g., for chronic-disease management) that is trained on real-world evidence from electronic medical records and health insurance claims. This casual recommender system, a set of machine-learning derived measures, can be used to suggest personalized treatment regimens. The automated causal recommendation engine described herein can be used for chronic-disease management, and is capable of assessing an arbitrarily large number of simultaneous treatment options across numerous patient sub-groups. Moreover, the causal recommender can be used to generate a ranked list of treatments based on real-world observed efficacy for each sub-population while controlling for an arbitrarily large number of confounders.

In one aspect, some implementations include a computer-implemented method for implementing a causal recommender for personalized disease treatment selection using machine learning. The method includes obtaining health trajectories for patients. Each health trajectory corresponds to a respective patient and represents a time-ordered series of health events for the respective patient. Each health trajectory may include at least one health condition, at least one treatment event and at least one index event. Each health trajectory includes a respective plurality of sub-trajectories. Each sub-trajectory includes a treatment event and ends at a respective index event.

The method also includes stratifying the sub-trajectories for each patient to form a plurality of stratified patient segments. In some implementations, each stratified patient segment corresponds to a separate and distinct health condition and includes the sub-trajectories for patients that have the health condition. In some implementations, stratification is not based only on separate and distinct health conditions. Any set of patient covariates (e.g., age, gender) is used to segment out the population. In some implementations, a series of if- then-else rules can be used for stratification. For example, these rules are obtained from clinicians or other health experts.

The method also includes performing, for each segment of the plurality of stratified patient segments, pairwise causal inference analysis on one or more treatments corresponding to the sub-trajectories of the respective segment, to estimate average treatment effect (ATE) values. Network meta-analysis is performed on the ATE values, thereby ranking the one or more treatments for each patient in the respective segment. In accordance with a determination that a respective patient has a health condition which could cause a set of treatments to be unsafe based on one or more clinical rules and the health trajectories, reranking the one or more treatments after excluding the set of treatments. The method also includes outputting treatment options for personalized disease treatment selection for a patient based on ranked treatments for the plurality of stratified patient segments. For example, a patient may have some comorbidities that make a recommendation contraindicated. For example, insurance claims data may show that prescription caused health issues in a given population.

In some implementations, performing the network meta-analysis includes constructing a densely connected network graph based on the ATE values, and performing a Network Meta-Analysis (NMA) (e.g., Bayesian Network Meta-Analysis, Frequentist) on the densely connected network graph. For example, for Bayesian-Network Meta-Analysis, a hierarchical random-effects model is used, according to some implementations. For example, each node of the densely connected network graph may correspond to a treatment, and may be connected to every other node via the measured ATE as an edge to obtain a densely connected network.

In some implementations, performing the Network Meta-Analysis (NMA) includes computing synthesized ATEs, for the ATE values, and computing the synthesized ATEs against a baseline treatment. For example, in a causal recommender for Diabetes, the baseline treatment may be set to Metformin because it is the first-line therapy for Type 2 Diabetes Mellitus or T2DM.

In some implementations, performing the Network Meta-Analysis (NMA) includes computing a Surface Under the Cumulative RAnking curve (SUCRA) score for each treatment. For example, samples may be drawn from the posterior predictive distributions of the trained model to compute Surface Under the Cumulative RAnking curve (SUCRA) scores for all treatments. Performing the Network Meta-Analysis (NMA) further includes ranking the one or more treatments according to the SUCRA curve. For example, ranking the one or more treatments according to the SUCRA curve may lead to the most effective treatment being assigned the highest rank. If a Frequentist approach is used for NMA, ranking can be done on the basis of p-values.

In some implementations, the pairwise causal inference analysis includes neural-network causal analysis to determine causal inference between each pair of treatments of the one or more treatments. For example, this step uses a neural-network-based propensity-score model for causal inference. In some implementations, the pairwise causal inference analysis estimates a total of N² unique ATE values per segment, where N is the number of treatments (e.g., 15,000) in the respective segment.

In some implementations, the pairwise causal inference analysis uses inverse probability of treatment weighting (IPTW) method, where patients in control and treatment arms are assigned weights equal to the inverse probability for getting the treatment they received.

In some implementations, stratifying the sub-trajectories includes grouping patients on the basis of clinical covariates in the health trajectories.

In some implementations, stratifying the sub-trajectories is performed by applying a machine learning algorithm on the health trajectories. In some implementations, the machine learning algorithm is an unsupervised k-means clustering that clusters similar patients based on treatments.

In some implementations, stratifying the sub-trajectories is performed by generating a bespoke recommender for each patient trained on a cohort of their k-nearest-neighbors.

In some implementations, stratifying the sub-trajectories includes splitting (sometimes called segmenting) the health trajectories into segments based on age, prior treatment, and comorbidity index values.

In some implementations, the method further includes selecting treatments that have at least a minimum cohort size to include in the one or more treatments. For example, a minimum cohort size may be a predetermined value, such as 30.

In some implementations, the method further includes: for each patient of the plurality of patients: identifying a respective treatment event and a respective index event in the health trajectory for the respective patient, wherein a respective index event is any clinical or health data point; and segmenting the health trajectory into a respective plurality of sub-trajectories such that each sub-trajectory includes a treatment event and ends at a respective index event. In some implementations, each sub-trajectory terminates in a pair of lab measurements (e.g., HbA1c lab measurements, two blood pressure events for Diabetes), and the method further includes: computing age of the respective patient, any comorbidities, and prior medication as of the date of the first lab of the pair of lab measurements in the sub-trajectory; and using current medication as the treatment for the patient corresponding to the sub-trajectory, for the period between the two labs of the pair of lab measurements. In some implementations, while segmenting the health trajectory, the method also includes excluding sub-trajectories where the duration between the lab pairs is not within a predetermined time period (e.g., less than 90 days or greater than 365 days). Two or more lab measurements occurring in a very short period of time may not be very different and causally attributing the small difference to the treatment may be problematic. Instead of excluding the sub-trajectories, labs that occur very close together (e.g., same day or same week) can be averaged. On the other hand, labs that occur too far apart are also problematic, because many of the confounders are measured as of the time of the first lab of the pair, and these confounders may have changed substantially when the period between the labs is too long. This can again lead to problems in causal attribution to the treatment. In some implementations, removing patients with a single lab measurement from the plurality of patients, prior to splitting the health trajectory. In some implementations, when the respective patient has multiple medications, the method further includes using a combination of the medications as the treatment for the respective patient for the period between the two labs of the pair of lab measurements.

In some implementations, the method further includes, for a new patient, recommending a personalized treatment option by identifying one or more sub-trajectories for a particular stratified patient segment that are most similar to the new patient's health trajectory. In some implementations, for new patient(s) (e.g., a patient whose previous medical history is unknown), recommendations are based on the strata to which they belong.

In another aspect, a system configured to perform any of the above methods is provided, according to some implementations.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1A is a flow diagram for a method for preparing data used to train a causal recommender system for personalized healthcare, according to some implementations.

FIG. 1B illustrates segmentation of each patient's health history into a series of temporal snapshots, according to some implementations.

FIG. 1C illustrates training data for a patient, according to some implementations.

FIG. 2 shows a schematic of a process used to train and/or evaluate the causal recommender system, according to some implementations.

FIGS. 3A and 3B show standardized differences between control and treatment cohorts covariates treated as confounders, according to some implementations.

FIG. 4 shows example results of network meta-analysis, according to some implementations.

FIG. 5 shows change in lab measurements for concordant and non-concordant cohorts for all clinical subgroups, according to some implementations.

FIG. 6 depicts graph plots showing sensitivity analysis, according to some implementations.

FIG. 7 shows a forest plot of Average Treatment Effect (ATE) values of treatments in a segment, according to some implementations.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

As described above in the Summary section, there is a need for a causal recommendation engine capable of assessing an arbitrarily large number of simultaneous treatment options across numerous patient sub-groups, to generate a ranked list of treatments based on real-world observed efficacy, for each sub-population, while controlling for an arbitrarily large number of confounders.

Traditionally, providers have relied on medical guidelines when prescribing drugs for chronic diseases (e.g., Type 2 Diabetes Mellitus). Such guidelines are prescriptive for first-line therapies, but if first-line therapies fail, the guidelines leave each physician to use their best judgement to select one option from sometimes several thousand options. For example, when the disease has been inadequately managed for a prolonged period despite following guidelines for first or subsequent lines of treatment, physicians oftentimes resort to informed guesswork to formulate treatments that will bring their patient's disease under control. This problem is exacerbated for combination therapies where many drug choices are available, and the number of treatment options becomes combinatorially large.

To supplement guideline-based practice, a causal recommender system for healthcare management is described herein. The system is trained on real-world evidence from electronic medical records and claims of a large number of patients (e.g., more than 100,000 patients with an A1C greater than 9%). In stratified sub-populations, the recommender is trained on more than ten thousand (e.g., 15,000) confounder-adjusted, case-control, observational studies between drug combinations (e.g., anti-hyperglycemic drug combinations), ranks treatment options based on their comparative efficacy using network meta-analysis, and returns sets of most effective medications for those sub-populations. In a retrospective study, after controlling for confounding, the causal recommender system found that individuals that followed recommendations lowered their levels of glycated hemoglobin by approximately 1% versus non-compliers. Causal recommender systems like the one described here are an important step towards achieving population-level glycemic control.

In some implementations, the automated causal recommender engine utilizes Balancing Covariates Automatically Using Supervision (BCAUS) to create covariate balance guarantees along with Inverse Probability of Treatment Weighting (IPTW) as the counter-factual generating causal framework, followed by a Network Meta-Analysis (NMA) to create rankings. Some implementations use modern deep learning to optimize these well-established methods. In some implementations, as a final step, a recommendation engine is employed on held-out data to retrospectively estimate the improvements to patient outcomes that would be seen if physicians used this causal framework to inform treatment decisions. Concordant prescriptions may be defined herein as those where a top-3 treatment for a patient (sub-group specific) is given, and non-concordant as any other option.

In some implementations, the management of glycemic control (HbA1C) is used for advanced, prior treatment-exposed, adult patients with type 2 diabetes as an illustrative use case, utilizing real-world evidence (e.g., claims, laboratory results, socio-demographics, from 1.2 million patients). Patients are divided into multiple clinical sub-populations (e.g., 10 groups) based on age, insulin dependence, and disease-burden, resulting in a large number of simultaneous trial arms (e.g., approximately 15,000 simultaneous trial arms). For this example, a treatment may be defined as the combination of one or more classes of antihyperglycemics, giving 364 unique treatments. After filtering down to treatments seen, at least 35 times in a particular sub-group, there are approximately 15,000 pairwise comparisons.

### Overview of Recommender Systems for Personalized Healthcare

Recommender systems have become ubiquitous in our online lives. We rely on them to select what movies to stream, what products to buy on websites, or which of our social media friends to follow. Such systems create personalized experiences and tailor their offerings to be most suitable for the particular characteristics of their target consumers. Additionally, when the number of available options may be potentially unlimited, they are effective at solving the “long-tail” problem by identifying choices that are less popular in the general population but may lead to increased consumer satisfaction in niche segments.

In healthcare, RCTs are conducted in small, well-controlled cohorts to evaluate safety and efficacy of medical treatments. In such trials it may be most common to compare a single treatment against a placebo, though some studies may have multiple arms where a few drugs are tested simultaneously. The physician in the clinic, outside of the idealized settings of an RCT, is faced with a different challenge: instead of deciding between placebo and treatment, the physician has to decide between several choices of approved drugs and, where combination therapy is indicated, how best to combine available drugs. Medical guidelines provide valuable support to ease this decision-making process. However, when options specified by guidelines have been exhausted, as may be the case for patients with long-standing chronic conditions, the physician may need to iterate between different treatment choices before finding the optimal one. The twin problems identified here i.e. seemingly endless choice and the lack of personalization, are well suited to be tackled by a recommender system.

Observational studies on retrospective healthcare data may provide additional evidence to support RCTs by determining the real-world efficacy of drugs. In such studies, covariate distributions between treatment arms can vary and it is essential to disambiguate between associative and causal effects of treatment. Variables that influence the treatment choice as well as the effect (referred to as confounders), if not properly accounted for, can blur the distinction between the two. Using causal inference studies, confounders are identified based on clinical inputs and/or user input, and used for model fitting, and subsequently inference purposes, according to some implementations. Treatment options are systematically identified and individuals between treatment arms are matched to simulate the controlled settings of an RCT and tease out the causal treatment effect from the observed effect. Observational studies also offer a unique opportunity to measure causal effects between treatments that haven't been compared directly in an RCT. By measuring relative effects of approved drugs, some implementations rank treatment options based on their comparative efficacy and find treatments that are optimal.

Meta-analytic studies combine results across different RCTs conducted for a particular control-treatment pair to compute a more robust estimate of the treatment effect. When multiple treatments exist, in some implementations, experimental evidence is represented as a connected graph where the nodes are the available treatments and the edges connect treatments which have been compared directly via (one or more) RCTs. In some implementations, a network meta-analysis (NMA) consolidates evidence across such a graph to indirectly compare treatments that have not been studied in an RCT and generate rankings based on the comparative efficacy of all available treatments relative to a baseline treatment.

According to some implementations, recommender systems for personalized medicine use recent advances in genomics and the large-scale digitization of patient medical records with concurrent advancements in machine learning and artificial intelligence. Unlike other application areas of recommender systems, the system described herein incorporates elements of causal modeling and uses evidence from multiple lines of investigation. In some implementations, the method described herein uses real-world data from health records of 100,000 patients to develop a recommender system for anti-hyperglycemic drugs that meets these requirements.

### Example Implementations

### Study Cohort Definition and Data Preparation

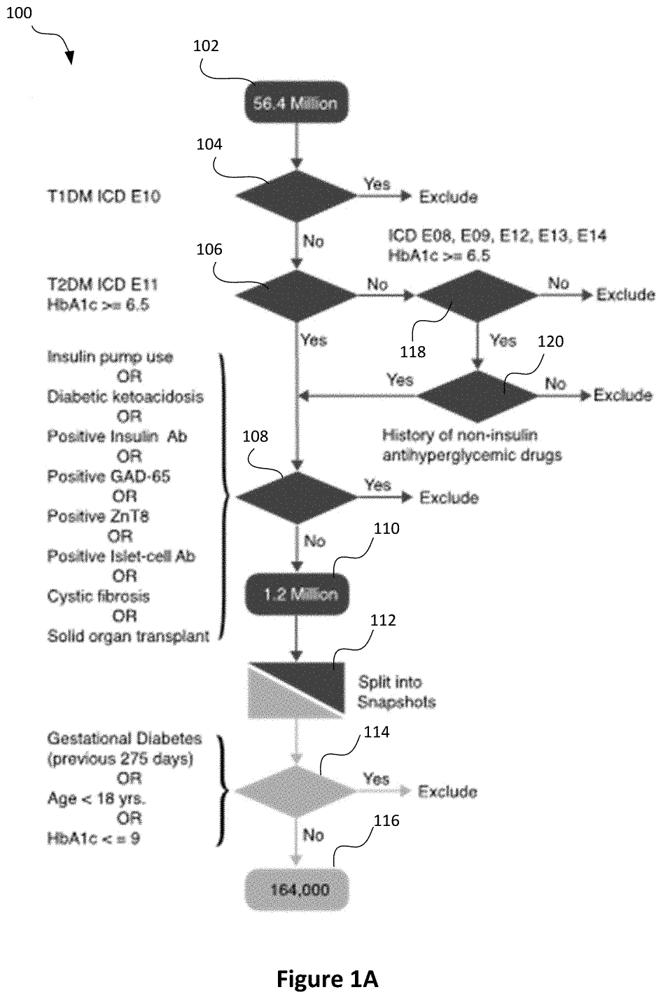

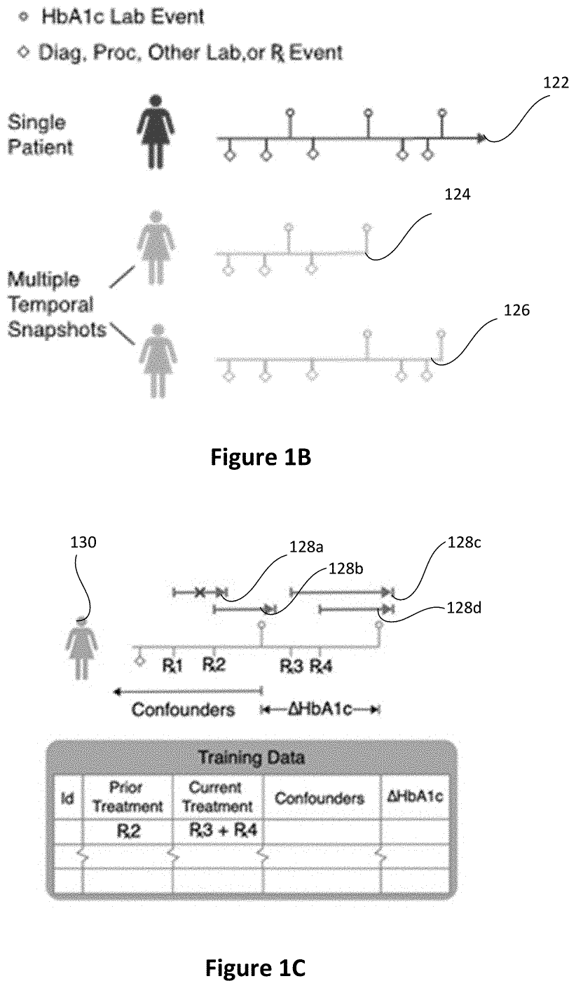

FIG. 1A is a flow diagram for a method 100 for preparing data used to train a causal recommender system for personalized healthcare, according to some implementations. As shown in FIG. 1A, to train the causal recommender system, health records 102 from a plurality of patients (e.g., 56.4 million patients) is extracted. The health records 102 include health trajectories for patients. Each health trajectory corresponds to a respective patient and represents a time-ordered series of health events for the respective patient. Each health trajectory includes at least one health condition, at least one treatment event and at least one index event. The health records 102 extracted may pertain to a predetermined time period (e.g., a 5-year time period that includes the time period between Dec. 1, 2014 and Jan. 1, 2020). Health records may pertain to patients of a specific age group (e.g., older than 18 years of age), patients with a specific disease (e.g., patients with Type II Diabetes Mellitus (T2DM)), and lab measurements (e.g., HbA1c above 9%). The health records may include insurance claims data (e.g., approximately 5 billion insurance claims) for diagnoses, procedures, and drug prescriptions or refills, and lab test results. Using the health records, in some implementations, the method includes using (104) one or more filters to first exclude all patients with some medical code (e.g., T1DM ICD E10). Next, in some implementations, the method uses (106) a filter to retain all patients with specific medical claims data or code (e.g., T2DM, ICD, E11, and HbA1c greater than or equal to 6.5). In some implementations, of the health records not retained by earlier filters, another filter may exclude (118) all patients who do not have some claims code (e.g., ICD E08, E09, E12, E14, HbA1c greater than or equal to 6.5). Further, of those not excluded by earlier filters (e.g., those patients who have ICD E08, E09, E12, E14, HbA1c greater than or equal to 6.5), a subsequent filter may then retain (120) those patients who have a history of a particular treatment (e.g., prescribed non-insulin anthyperglycemic drugs). Of the patients retained by earlier filters, a subsequent filter may further exclude (108) any patient using a specific treatment option (e.g., patients using an insulin pump, patients with diabetic ketoacidosis, positive insulin antibody (Ab), positive GAD-65, positive ZnT8, positive islet-cell Ab, cystic fibrosis, and/or solid organ transplant). In this way, a subset 110 (e.g., 1.2 million patients) of the plurality of patients is extracted. For example, the subset 110 includes patients with Type II Diabetes Mellitus (T2DM) using the filtering steps described herein. In some implementations, the method 100 and respective filters therein may be designed to distinguish between different types of a health condition (e.g., three sub-types of diabetes). In some implementations, the subset 110 is split (112) into snapshots (further described below) using (114) a subsequent filter. For example, of the 1.2 million patients, a filter may exclude all patients with gestational diabetes in the previous 275 days, less than 18 years of age or an HbA1c less than or equal to 9. It is noted that the example time periods and/or health conditions are only provided for illustration purposes, and can be easily parameterized, in various implementations.

In some implementations, the health records 102 pertain to data spanning several years, representing the health status of patients over a specific period of time. For example, the health records 102 of 56.4 million members extracted may change over the several years the system 100 collected data. Some implementations automatically and/or continuously collect healthcare information. Some implementations use manually input data or augmented data. The health records 102 of patients may have new diagnoses, medications may be discontinued, and/or new medications may be administered.

To properly account for this evolution, each patient's health history may be segmented into a series of temporal snapshots (also referred to herein as sub-trajectories, e.g., snapshots 122, 124, 126), as shown in FIG. 1B, according to some implementations. As described above, each health trajectory for a patient may include at least one health condition, at least one treatment event and at least one index event. Each health trajectory includes a respective plurality of sub-trajectories. Each sub-trajectory includes a treatment event and ends at a respective index event. According to some implementations, an index event is any clinical or health data point. In some implementations, each snapshot is terminated in a pair of lab measurements (e.g., HbA1c lab measurements. For example, the temporal snapshot 122 may pertain to a single patient. The temporal snapshots 124 and 126 may also pertain to a single patient, where the temporal snapshot 126 shows a longer evolution of the patients health record. Individuals with only a single HbA1c lab reported may be excluded from the training set. In some implementations, only snapshots where the duration between the lab pairs was between 90 to 365 days may be retained. In some implementations, only snapshots where the duration between the lab pairs was less than 90 days may be retained. In some implementations, snapshots where the duration was longer than 365 days may be retained. The age of the patient in a particular snapshot and any attributes treated as confounders may be computed as of the date of the first lab of the pair.

Referring next to FIG. 1C, a patient may be considered to have been treated by a particular anti-hyperglycemic drug at the time of a given HbA1c lab event, if it was prescribed prior to the lab and if the number of days of supply 128a, 128b, 128c and 128d extended past the lab date. For example, patient 130 may be treated by particular anti-hyperglycemic drugs for number of supply days 128a, 128b, 128c and 128d, where the longest number of supply days may be 128c. When multiple such drugs exist, some implementations consider the patient to be treated by the combination of these drugs. As indicated in training data for patient 130 in FIG. 1C, the current treatment received is a combination of R3 and R4, whereas the prior treatment for patient 130 may have been R2 by itself. Diabetes drugs may be identified only by their class names (e.g., SGLT2 Inhibitor, GLP-1 Agonist) and non-diabetes drugs may be excluded. The causal recommender system may be trained on this pseudo-population of patient snapshots, according to some implementations. The age of the patient in a particular temporal snapshot (e.g., the temporal snapshots 122, 124 and 126) and any confounding covariates may be computed as of the date of the first lab of the pair of lab measurements, according to some implementations. In some implementations, prior treatment is the regimen used to treat the individual in the period prior to the observation window between the two labs. In some implementations, diabetes drugs may be identified only by their drug class names and non-diabetes drugs may be excluded.

Table 1 shown below provides example mean and standard deviation for Continuous Confounding Variables (CCV) of the health records 102 at the patient and snapshot levels, according to some implementations. For treatment personalization, individuals may be stratified based on their age and Charlson Comorbidity Index (CCI) into five segments. Each segment may be further subdivided on the basis of the presence or absence of insulin in the individual's prior treatment regimen.

TABLE 1
Mean & Std Dev for Continuous Confounding Variables (Patients)

| Confounder | Train: Mean ± Std Dev | Test: Mean ± Std Dev |
|---|---|---|
| Baseline HbA1c | 10.5 ± 1.4 | 10.5 ± 1.4 |
| ZCTA % | 59159 ± 22930 | 58847 ± 22727 |
| MedianIncome | | |
| ZCTA % White | 62.3 ± 23.2 | 62.0 ± 23.2 |
| ZCTA % Native Am. | 0.5 ± 1.0 | 0.5 ± 1.0 |
| ZCTA % Black | 16.2 ± 20.0 | 16.4 ± 20.1 |
| ZCTA % Asian | 7.6 ± 10.8 | 7.6 ± 10.8 |
| Age | 55.4 ± 10.9 | 55.3 ± 10.6 |
| Creatinine Lab | 0.9 ± 0.3 | 0.9 ± 0.3 |
| EGFR Lab | 94.1 ± 23.4 | 94.2 ± 23.1 |

Table 2 shown below provides example summary statistics for counts for binary confounding variables defined, according to some implementations.

TABLE 2
Counts for Binary Confounding Variables (Patients)

| Confounder | Train: % Present | Test: % Present |
|---|---|---|
| Renal Disease | 11.5 | 11.0 |
| Cancer | 4.8 | 4.8 |
| Metastatic Carcinoma | 0.7 | 0.6 |
| Connective Tissue Disease | 2.3 | 2.2 |
| Dementia | 0.9 | 0.8 |
| Paraplegia and Hemiplegia | 1.0 | 0.9 |
| Cerebrovascular Disease | 7.9 | 7.5 |
| Chronic Pulmonary Disease | 17.9 | 17.6 |
| Peptic Ulcer Disease | 1.4 | 1.3 |
| Diabetes w Complications | 40.1 | 40.7 |
| Diabetes w/o Complications | 94.6 | 95.2 |
| Mild Liver Disease | 11.9 | 11.4 |
| Severe Liver Disease | 0.6 | 0.6 |
| Obesity | 40.7 | 40.8 |
| Hypoglycemia | 2.4 | 2.3 |
| ASCVD | 14.7 | 14.5 |
| Peripheral Vascular Disease | 7.8 | 7.8 |
| Heart Failure | 11.6 | 11.5 |
| End-Stage Renal Disease | 0.7 | 0.6 |
| Dialysis | 0.3 | 0.3 |
| Chronic Kidney Disease | 13.2 | 12.7 |
| Fructosamine Test | 1.0 | 0.9 |
| Gastroparesis | 0.5 | 0.5 |

Table 3 shown below provides an example for training datasets split into 10 segments based on age, prior use of insulin and comorbidity index values, according to some implementations. The number of available treatments, based on NMA rankings, varies for each group and may be typically higher for larger cohort sizes. Concordant percent values indicate the percent of each group that received a drug recommended in either the top 3 rankings (top-3) or the top 4-10 rankings (top 4-10) or anywhere between position 11 and a drug not in the NMA rankings list (bottom). The number of patients on a top-3 most-effective treatment is extremely small, ranging from less than 1% in the most common subgroups to approximately 10% in the subgroups containing the most extremely sick.

TABLE 3
Summary Stats per Stratum

| | Prior | | Comorbidity | Train: No. of | Train: No. of | Train: No. of | | | |
|---|---|---|---|---|---|---|---|---|---|
| Segment | Insulin | Age | Index | Patients | Snapshots | Treatments | Top-3 | Top 4-10 | Bottom |
| 1 | 0 | <65 | <=2 | 32757 | 43532 | 69 | 1% | 6.8% | 92.2% |
| 1 | 1 | <65 | <=2 | 7025 | 10734 | 43 | 2.3% | 6.6% | 91.1% |
| 2 | 0 | <65 | >=3 | 12649 | 17072 | 50 | 1.5% | 9.1% | 89.4% |
| 2 | 1 | <65 | >=3 | 6614 | 10447 | 43 | 1.5% | 6.8% | 91.7% |
| 3 | 0 | <65 | >=5 | 5450 | 7331 | 37 | 3.6% | 7.4% | 89% |
| 3 | 1 | <65 | >=5 | 4343 | 7180 | 35 | 3.8% | 9% | 87.2% |
| 4 | 0 | >=65 | <5 | 5486 | 7177 | 30 | 2.5% | 11% | 86.5% |
| 4 | 1 | >=65 | <5 | 2033 | 2931 | 16 | 8.5% | 36% | 55.5% |
| 5 | 0 | >=65 | >=5 | 2709 | 3471 | 18 | 9.8% | 37.5% | 52.7% |
| 5 | 1 | >=65 | >=5 | 2264 | 3425 | 23 | 3.7% | 25.7% | 70.6% |

| | | | |
|---|---|---|---|
| Total | 68888 | 113300 | 364 |

The example described above stratified patients into 10 segments. A higher degree of personalization is possible by using other strategies. Unsupervised learning via k-means clustering can be used to discover subsets of related individuals. Higher degree of personalization can be achieved by defining cohorts consisting of the k-nearest neighbors of a seed member. Some implementations implement personalization by ranking according to Individual Treatment Effects (ITEs) instead of Average Treatment Effects. Such methods will improve treatment selections considerably when tested on retrospective data. Some implementations trade off clinical transparency for treatment selection options.

### Example Causal Recommender

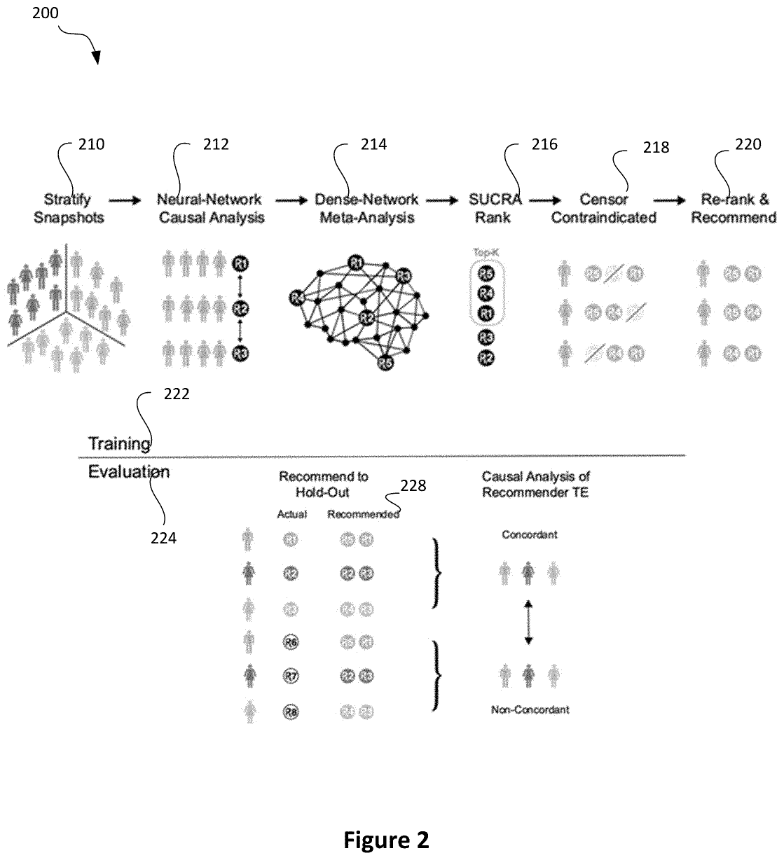

FIG. 2 shows a schematic of a process 200 used to train and/or evaluate the causal recommender system, according to some implementations. As shown in FIG. 2, during a training phase 222, the process 200 includes stratifying (210) snapshots to divide a patient population. For example, the population may be divided into a predetermined number of subgroups (e.g., ten clinical subgroups), based on age, number of comorbidities, and/or prior insulin use (e.g., as shown in Table 3 above). In some implementations, the population may be further stratified. In some implementations, the stratified snapshots may have fewer divisions than what is shown herein. Subsequently, for each clinical subgroup, treatments may be selected (e.g., all treatments with cohort size >35 may be selected), and case-control observational studies are performed, comparing every treatment with every other treatment using a neural-network-based propensity-score model for causal inference (212), according to some implementations. In some implementations, a cohort size greater than 30 may be selected. For example, a total of 14,840 neural networks may be trained in total, one per observational study. As another example, a total of 15,198 neural networks may be trained, one for each case-control observational study. The propensity-score models may be used to estimate pairwise Average Treatment Effects (ATE) and associated confidence intervals, according to some implementations. These values may then be used to construct a graph, where each node representing a treatment is connected to every other node via the measured ATE as an edge to obtain a densely connected network. Network meta-analysis is performed (214) subsequently. For example, Network Meta-Analysis (NMA) may be performed to compute network-synthesized ATEs against a baseline treatment which may be set to Metformin (the first-line therapy for T2DM). Subsequently, treatments are ranked (216, e.g., using the treatments' Surface Under the Cumulative RAnking curve (SUCRA) scores) and a top predetermined number of treatments (e.g., Top-K=3) are returned to each member of the subgroup (e.g., treatments may be ranked in decreasing order of the SUCRA score as recommendations for the segment). In some implementations, patient's health history is checked for health conditions which would cause any of the returned treatments to be unsafe, and such contraindicated treatments are censored (218). A series of if-then clinical rules (see e.g., Table 4) may be used to remove such contraindicated treatments. This process may be repeated for all stratified segments in the training dataset.

In some implementations, patient snapshots are split into training (80%) and evaluation (20%) datasets, such that in each dataset the relative sizes of the segments are the same. To gauge the efficacy of the causal recommender system, steps shown in an evaluation phase 224 may be performed on the trained model (model trained using the steps in training phase 222, described above). Recommendations 228 may be generated for each patient using the method described above and their change in lab measurements (e.g., HbA1c) may be recorded. The change in the lab measurements (e.g., HbA1c) between concordant cohort (where the treatment matched one of the recommendations) and non-concordant cohort may be compared in a causal inference setting to estimate the ATE of the causal recommender system. This metric is used to validate the model and anticipate, using a retrospective analysis, the additional improvement in HbA1c the recommender system would have over the standard-of-care if such a system may be deployed in a prospective study.

### Example Causal Inference Techniques

Among causal inference techniques, the most popular by far are ones based on propensity-score modelling. A propensity-score model is a binary classifier that is trained to predict the probability or propensity for receiving a particular intervention (either control or the treatment under study), using covariates of the cohort as input. Several variants of propensity score modelling exist. The inverse probability of treatment weighting (IPTW) method may be used, where individuals in control and treatment arms are assigned weights equal to the inverse probability for getting the treatment they received. If the propensity model is correctly specified, this weighting creates pseudo-populations in the two arms that are matched in every covariate except the intervention under study. The estimated treatment effect can then be causally attributed to the intervention. In some implementations, the typical IPTW workflow includes three steps: i) the propensity model is trained using assigned treatments as targets, ii) standardized mean differences are computed between the inverse propensity weighted covariates of the two arms to test for removal of confounder imbalance, and iii) the ATE is computed as a weighted average of the outcomes. If covariate imbalance has not been sufficiently removed in step ii, the propensity model is deemed to be incorrectly specified and the classifier has to be retrained by varying model definition or via data transformations till the two arms are sufficiently balanced. Most often, observational studies are performed to compare a single treatment against a single control and this iterative procedure suffices. However, when thousands of studies need to be performed, as is the case for the causal recommender, this approach becomes infeasible.

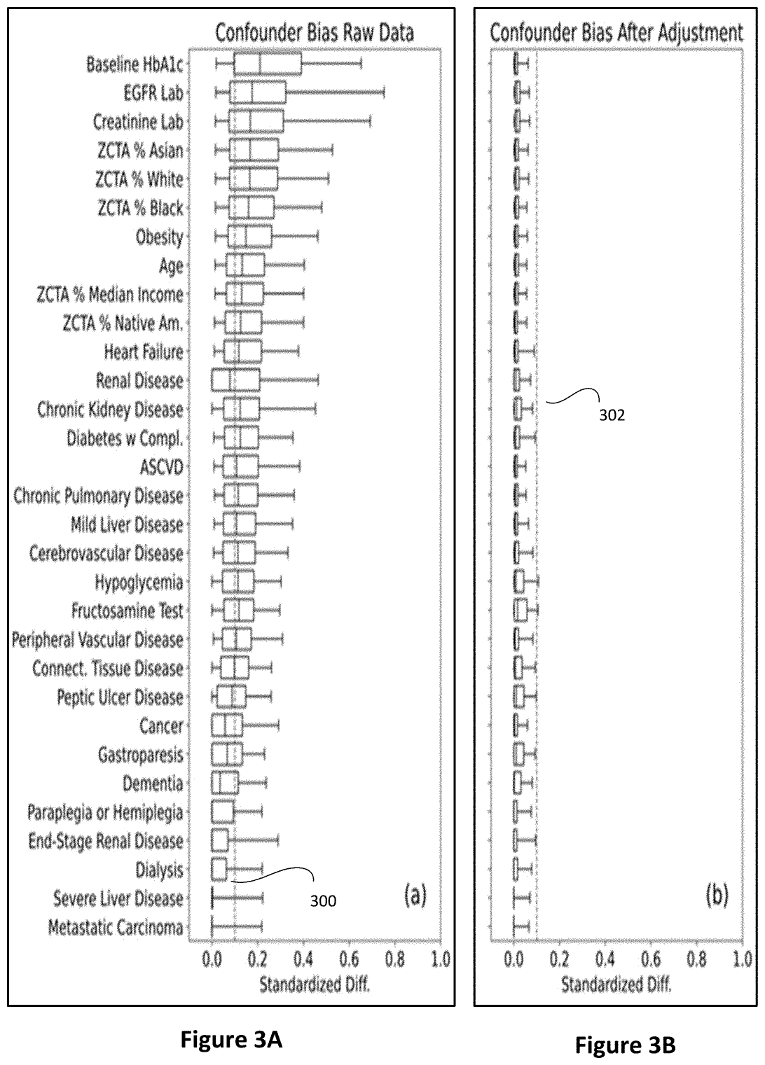

Some implementations use a technique called BCAUS (Balancing Covariates Automatically Using Supervision) to perform causal analysis in massive multi-arm studies that is well suited for the causal recommender. As shown in FIG. 3A, BCAUS consists of a neural-network propensity model that is trained using a joint loss given by [symbol] _{TOTAL}= [symbol] _{BCE}+νμ [symbol] _{BIAS}, according to some implementations. The first term, [symbol] _{BCE} is a binary cross-entropy loss which penalizes incorrect treatment assignment, while the second, [symbol] _{BIAS} is a loss term which explicitly tries to minimize imbalance between inverse probability weighted covariates. Details of extraction and transformation processes, including causal analysis with BCAUS, are described below, according to some implementations. For each pairwise comparison between treatments in the causal recommender, a separate BCAUS model may be trained. The outputs of trained models may be used to compute inverse probability scores and estimate ATEs. A bootstrapping procedure may be used to compute standard errors and confidence intervals. The input data for NMA consisted of the estimated ATEs and standard errors.

To ascertain if all 10,000 propensity models are correctly specified, for each observational study, the standardized mean difference (SMD) between control and treatment groups for every confounder was computed prior to and after inverse-propensity-based adjustment. A commonly accepted rule-of-thumb is to consider a confounding covariate as sufficiently balanced if the SMD is below a threshold value of 0.1. The plots in FIG. 3B show SMDs between covariates prior to BCAUS training and adjustment, according to some implementations. Each box and whisker represents the distribution of SMDs for all 10,000 observational studies. For many covariates, the imbalance significantly exceeds the threshold value 204. As shown in FIG. 3B, after adjustment by inverse probability weights from BCAUS, covariate imbalance reduces substantially suggesting that a majority of the trained models are well specified. For each observational study, the number of balanced covariates is counted and ATEs from the study are included in the NMA only if all 31 covariates are balanced, according to some implementations. Of the 15,198 models, 10,251 models had all 31 covariates balanced and these observational studies are included in the NMA.

FIGS. 3A and 3B show standardized differences between control and treatment cohorts for all covariates (31 covariates in the example) treated as confounders, according to some implementations. Each box plot shown in FIGS. 3A and 3B summarizes covariate imbalance from 14,840 observational studies. Box edges show the lower and upper quartiles and line shows the median. Lower and upper whiskers show the 5th and 95th percentiles respectively. Threshold lines 300, 302 may be at standardized difference equal to 0.1, which is conventionally used as a threshold in causal inference studies to determine if a covariate has been sufficiently balanced. FIG. 3A shows bias in raw data without adjustments, while FIG. 3B shows data after covariates have been adjusted using inverse propensity weights from the neural network propensity-score models, according to some implementations. In the drawings, EGFR refers to Estimated Glomerular Filtration Rate, ZCTA refers to Zip Code Tabulation Area, Native Am refers to Native American, ASCVD refers to Atherosclerotic Cardiovascular Disease, and Diabetes w Compl. refers to Diabetes with complications. FIGS. 3A and 3B show that it is possible to control for confounding across thousands of simultaneous treatment arms.

### Example Causal Analysis with BCAUS

As indicated above, in some implementations, the BCAUS model is trained using the joint loss [symbol] _{TOTAL}= [symbol] _{BCE}+νμ [symbol] _{BIAS}. Here, μ is the scalar ratio of [symbol] _{BCE} to [symbol] _{BIAS} that is detached from the computation graph. The relative contribution of each loss component is tuned using hyperparameter, ν. The cross-entropy loss is calculated as:

$${ℒ}_{B C E} = {\sum }_{\dot{t}} {t}^{( i )} log ( {p}^{( \dot{ι} )} ) + ( 1 - {t}^{( i )} ) log ( 1 - {p}^{( i )} ) \qquad ( S 1 )$$

In the above equation, t⁽ⁱ⁾∈{0, 1} is the treatment given to individual i. To compute the bias loss, the propensity score p⁽ⁱ⁾ is used to compute the inverse probability weight (IPW) using the following equation:

$${w}^{( i )} = \frac{{t}^{( i )}}{{p}^{( i )}} + \frac{1 - {t}^{( i )}}{1 - {p}^{( \dot{ι} )}} \qquad ( S 2 )$$

The mean squared error of the M covariates weighted according to the equation (S2) is used to calculate the bias loss, according to the following equation:

$${ℒ}_{BIAS} = \frac{1}{M} {\sum }_{j = 1}^{M} {( \frac{{\sum }_{\dot{t}} {t}^{( i )} {w}^{( i )} {x}_{j}^{( i )}}{{\sum }_{i} {t}^{( i )} {w}^{( i )}} - \frac{{\sum }_{i} ( 1 - {t}^{( \dot{ι} )} ) {w}^{( i )} {x}_{j}^{( \dot{ι} )}}{{\sum }_{\dot{t}} ( 1 - {t}^{( i )} ) {w}^{( i )}} )}^{2} \qquad ( S 3 )$$

The two terms in the equation above represent the weighted means of the covariates for the treatment and control groups respectively. To assess balance, the standardized mean difference Δⱼ for each covariate j is computed according to the following equation:

$${Δ}_{j} = \frac{\vert {\overline{x}}_{j , treatment} - {\overline{x}}_{j , c o n t r o l} \vert}{\sqrt{( {s}_{j , treatment}^{2} + {s}_{j , control}^{2} ) / 2}} \qquad ( S4 )$$

In the equation (S4) shown above, x̅ⱼ is the weighted mean of xⱼ and sⱼ² is its weighted variance with weights assigned according to the equation (S3). The standardized mean difference can also be defined for the raw data without the weights, in which case xⱼ and sⱼ² represent the unweighted mean and variance respectively.

In some implementations, the BCAUS model is implemented in Python using the PyTorch neural networks library. Each BCAUS model consists of two hidden layers with the number of neurons in each layer set to twice the number of input covariates. Rectified Linear Units (ReLU) activation is used for all layers except the last layer consisting of single neuron which uses sigmoid activation. The learning rate is set to 0.001, the hyperparameter ν is set to 4 and the networks are trained for 1000 epochs. An early-stopping procedure is implemented where training terminated if all covariates remained balanced (i.e. standardized mean difference <0.1) for more than 10 epochs.

For each clinical subgroup, all treatments with more than 35 treated individuals are chosen and BCAUS models are trained comparing every treatment with every other treatment. For a treatment pair i and j, the estimated ATE values should be antisymmetric, i.e., ATEᵢⱼ=−ATEⱼᵢ, and for n treatments, n(n−1)/2 pairwise comparisons should suffice. However, since the propensity scores output by BCAUS are not calibrated probabilities, a small deviation from this asymmetric property (with differences much smaller than the standard error) is observed in practice. Therefore, ATEᵢⱼ and ATEⱼᵢ are computed separately and a total of n(n−1) BCAUS models are trained. Prior to training, all continuous covariates in each clinical subgroup were Z-scored to have zero mean and unit standard deviation. Propensity scores trimming is applied at the 0.01 level (e.g., propensity scores below 0.01 are set to 0.01 and those above 0.99 are set to 0.99). This ensured that no individual received an inverse propensity weight >10. A bootstrapping procedure is used to estimate the standard error for the ATE values. Inverse propensity weighted outcomes are picked at random and with replacement from the dataset and ATEs are computed between control and treatment individuals in each draw. The standard deviation of ATE values across 100 draws is reported as the standard error. ATE values and their standard errors for all pairwise treatment combinations are computed for each clinical subgroup and Network Meta-Analysis (NMA) is performed using example techniques described below, according to some implementations.

### Example Network Meta-Analysis

An ATE value measured via a direct causal comparison between two treatments has to be consistent with values that are indirectly estimated (under the transitivity assumption) by comparing each treatment of the pair with intermediary treatments and then computing differences, according to some implementations. To build a consolidated and self-consistent view of the evidence, a densely connected network graph is constructed for each stratified segment where every treatment node was connected with every other treatment node. Edges representing observational studies where all confounding covariates are not balanced are trimmed and NMA is performed over the resultant graph. Heterogeneity in the treated populations is accounted for by using a random-effects hierarchical model, uninformative priors are set, and a Markov Chain Monte Carlo (MCMC) sampling procedure was used to construct posterior distributions of ATE values for all treatment pairs. To determine relative ranks, samples are drawn from the posterior predictive distributions of ATEs of all treatments compared against Metformin, which are treated as the baseline treatment. For each draw, treatments are ranked in ascending order of ATE values (i.e., higher ranks for more negative values), and a mean rank is computed for each treatment across all draws. This mean rank is normalized to compute the SUCRA score. Treatments are ranked in descending order of SUCRA scores such that the treatment that reduced HbA1c by the largest amount relative to Metformin had the highest rank. This ranked list of treatments is returned to all members of the segment.

To illustrate Network Meta-Analysis, an example implementation is described herein, according to some implementations. In some implementations, Network Meta-Analysis is performed with a Python code developed using the PyMC3 probabilistic programming library. The network graph is encoded as a hierarchical, mixed-effects model:

ATEᵢⱼ˜Normal(δᵢⱼ,seᵢⱼ²) (S5)

δᵢⱼ=dᵢⱼ+τNormal(0,1) (S6)

dᵢⱼ=dᵢ−dⱼ (S7)

τ˜HalfCauchy(5) (S8)

dᵢ˜Normal(1,15*max(|ATE|)) (S9)

In the equations shown above, ATEᵢⱼ is the ATE value measured by comparing treatment i against treatment j and seᵢⱼ is the corresponding standard error, dᵢ is the ATE of treatment i relative to the baseline treatment with d_{baseline}=0. Uninformative priors are set for τ (the hierarchical standard deviation) and dᵢ with the standard deviation for the sampling distribution of the latter set to 15 times the maximum absolute value of measured ATEs. A non-centered parameterization is chosen for the model, because Markov Chain Monte Carlo (MCMC) samplers have difficulties sampling from the “Neal's funnel” that can lead to divergent trajectories and biased results. A No U-Turns Sampler (NUTS) is tuned with 10,000 warm-up steps and 100,000 samples are drawn from 4 chains that are run simultaneously. The tuning samples and the first 50,000 samples in each chain are discarded. To compute SUCRA scores, 200,000 samples are drawn from the posterior distribution of dᵢ and treatments are ranked for each draw. The SUCRA score for treatment i is calculated as:

$$S U C R {A}_{i} = \frac{n - 1 - 〈 {R}_{i} 〉}{n - 1} \qquad ( S 10 )$$

In the equation (S10), [symbol] Rᵢ [symbol] is the mean rank for treatment i across all draws and n is the number of treatments (Rᵢ∈[0, n−1]). Posterior samples are used to compute the mean and 94% credible intervals for ATE values dᵢ of all treatments relative to Metformin, the baseline treatment.

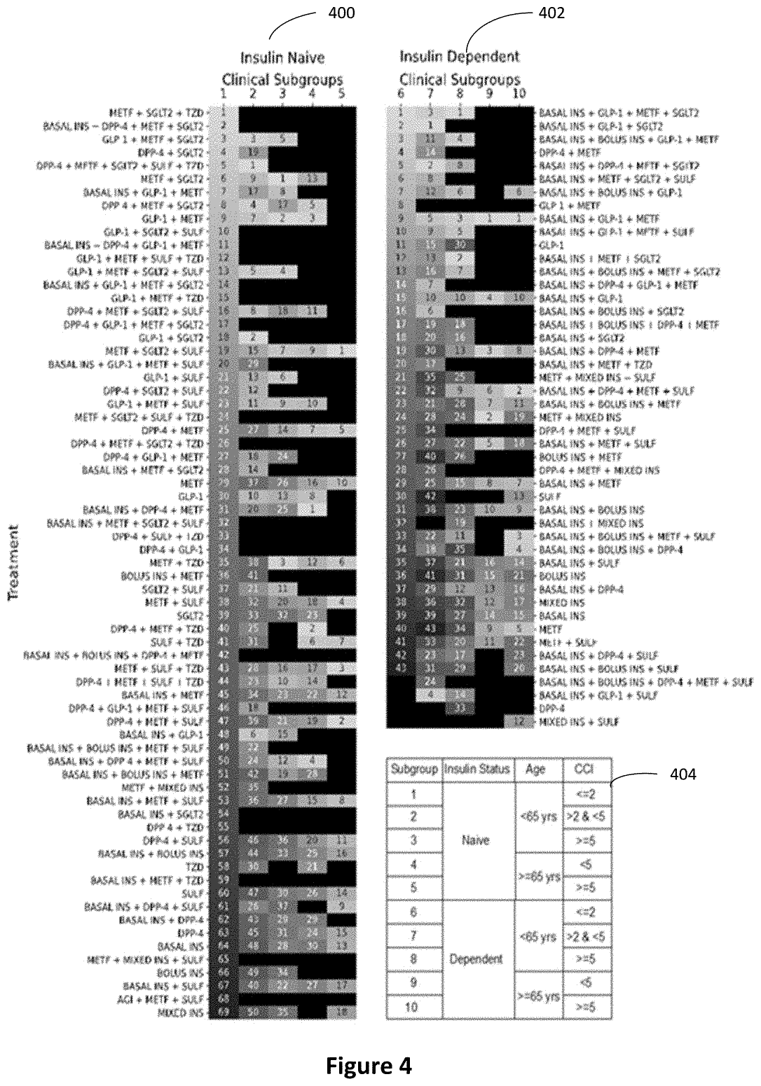

FIG. 4 shows example results of network meta-analysis, according to some implementations. In FIG. 4, treatments are ranked according to their SUCRA scores for each clinical subgroup. Left panel 400 shows Clinical Subgroups where prior treatment did not contain Insulin. Right panel 402 shows Clinal Subgroups where prior treatment contained Insulin. Lower right table 404 shows subgroup definitions. In FIG. 4, INS refers to Insulin, GLP-1 refers to Glucagon-Like Peptide-1 Receptor Agonist, SULF refers to Sulfonyurea, METF refers to Metformin, MEGL refers to Meglinitide, DPP-4 refers to Dipeptidyl Peptidase 4 Inhibitor, TZD refers to Thiazolidinedione, SGLT2 refers to Sodium-Glucose Transport Protein 2 Inhibitor, AGI refers to Alpha-Glucosidase Inhibitor, and CCI refers to Charlson Comorbidity Index. FIG. 4 shows the ranked list of effective drugs for each of 10 patient clinical sub-populations. As can be seen, the efficacy of each treatment varies considerably across clinical sub-populations.

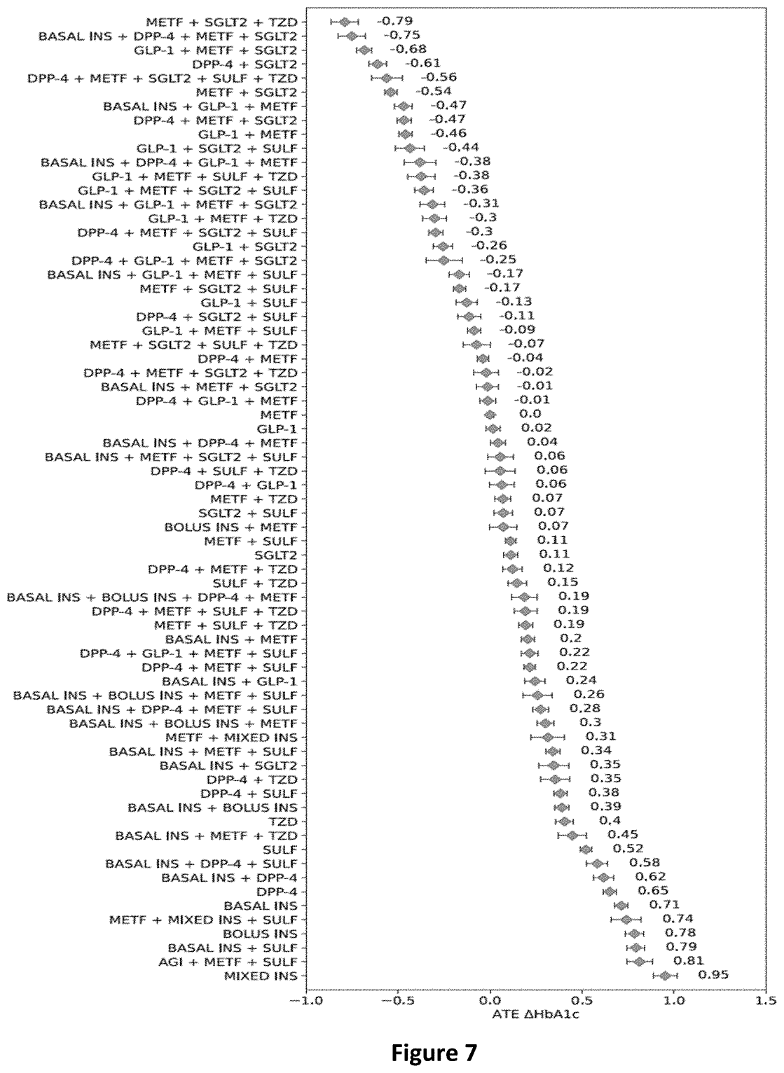

FIG. 7 shows a forest plot of ATE values of all treatments in a segment, according to some implementations. For comparison, direct ATE values are also shown of these treatments relative to Metformin measured via causal analysis but without NMA. Observe that consolidating evidence greatly improves the confidence in estimated ATEs which leads to more robust rankings. FIG. 4 shows a league table comparing the top 5 treatments in this segment against each other, according to some implementations. ATE values from NMA reported in the insulin dependent clinical subgroups graph in FIG. 4 are consistent with those reported in insulin naïve clinical subgroups graph of FIG. 4, showing that the transitivity property is observed to be satisfied.

### Causal Recommender Evaluation

The efficacy of a recommender engine like the one described here, may be measured by deploying it in an RCT, where members of a “treatment” cohort get optimized recommendations from the engine while the “control” cohort gets the standard-of-care treatment as decided by a physician without access to the recommendations. Any differences in measured outcomes (e.g., reductions in HbA1c) can then be causally attributed to the recommender engine. In the absence of such a randomized trial, it is still possible to approximately estimate the causal effect of the recommender by performing an observational study with retrospective data. To do this, the held-out test dataset may be used as a study cohort. For each patient snapshot in this set, ranked treatment recommendations may be generated depending on the stratified segment to which the snapshot belonged. For individuals with certain health conditions, one or more of the drug classes in a particular treatment regimen may be contraindicated. A set of safety filters may be used to check if such contraindicated drugs are present in the returned list of combination treatments, and when present, the entire treatment is removed from the list. An example list of the safety filters applied is shown below, according to some implementations.

### Example Safety Filters

In some implementations, the health history of each individual is analyzed and any treatments which contained contraindicated drugs are removed from the set of recommendations. An example list of safety filters used to identify contraindicated drugs is shown in Table 4, according to some implementations.

TABLE 4
Example Safety Filters

| | | Lookback Period (as of |
|---|---|---|
| Rule | Diagnosis Codes | first lab of Snapshot) |
| If Heart Failure then never | I50 | Full |
| THIAZOLIDINEDIONES | I97.13 | |
| | T86.32 | |
| | I09.81 | |
| | I43 | |
| | I13 | |
| | I11.0 | |
| | I27.29 | |
| | I26.09 | |
| | A52.06 | |
| | A52.03 | |
| | I25.5 | |
| | I42.0 | |
| | I42.5 | |
| | I42.6 | |
| | I42.7 | |
| | I42.8 | |
| | I42.9 | |
| If Liver Failure then never | K70.3 | 365 days |
| THIAZOLIDINEDIONES | K70.4 | |
| | K71 | |
| | K71.1 | |
| | K71.2 | |
| | K71.7 | |
| | K72 | |
| | K74 | |
| | K75.4 | |
| | K76.3 | |
| | K75.9 | |
| | K76.2 | |
| | K76.7 | |
| | K91.82 | |
| If Bladder Cancer then never | Z80.52 | Full |
| THIAZOLIDINEDIONES | Z85.51 | |
| | C67 | |
| | C79.11 | |
| If Kidney Disease or eGFR <45, then | I120 | 365 days |
| never start METFORMIN | I131 | |
| | N032 | |
| | N033 | |
| | N034 | |
| | N035 | |
| | N036 | |
| | N037 | |
| | N052 | |
| | N053 | |
| | N054 | |
| | N055 | |
| | N056 | |
| | N057 | |
| | N18 | |
| | N19 | |
| | N250 | |
| | Z490 | |
| | Z491 | |
| | Z492 | |
| | Z940 | |
| | Z992 | |
| If on INSULIN, then recommended | | |
| treatment should always contain | | |
| INSULIN | | |
| If Thyroid C cell tumor or Multiple | E31.2 | Full |
| Endocrine Neoplasia then never GLP-1 | | |
| AGONISTS | | |
| If Fournier's Gangrene then never | N49.3 | Full |
| SGLT2 INHIBITOR | | |
| If Lactic Acidosis then never | E87.2 | 365 days |
| METFORMIN | | |
| If Gastroparesis then never GLP-1 | K31.84 | 365 days |
| AGONIST | | |
| If Thyroid Cancer or a history of | C73 | Full |
| Medullary Thyroid Cancer then never | | |
| GLP-1 AGONISTS | | |
| If Pancreatitis then never GLP-1 | K85 | Full |
| AGONISTS | | |
| If Pancreatic Cancer then never GLP-1 | C25 | Full |
| AGONISTS | | |
| If Acute Kidney Failure then never GLP- | N17 | Full |
| 1 AGONISTS | | |
| If ASCVD then never | I20 | Full |
| SULFONYLUREAS | I21 | |
| | I22 | |
| | I23 | |
| | I24 | |
| | I25 | |
| | I7090 | |
| If Pancreatitis then never ′DPP-4 | K85 | Full |
| INHIBITORS | | |

After censoring contraindicated treatments, the Top-K=3 of the remaining treatments are returned for each patient snapshot. If the current treatment of a patient matched one of the recommendations, the patient may be considered to be concordant with the recommendation. Roughly ˜5% of the patients were found concordant, which implied that a large majority of patients were being treated by regimens that were less than optimal. To determine the causal effect of the recommender, a BCAUS propensity model was trained using the same confounding covariates as before but considering the concordant cohort as the treatment cohort that is treated with the recommender, and the non-concordant cohort as the control group. Inverse propensity weights were used to adjust the outcomes (difference in lab measurements, e.g., ΔHbA1c) and the ATE of the causal recommender was estimated. Results are summarized in Table 2.

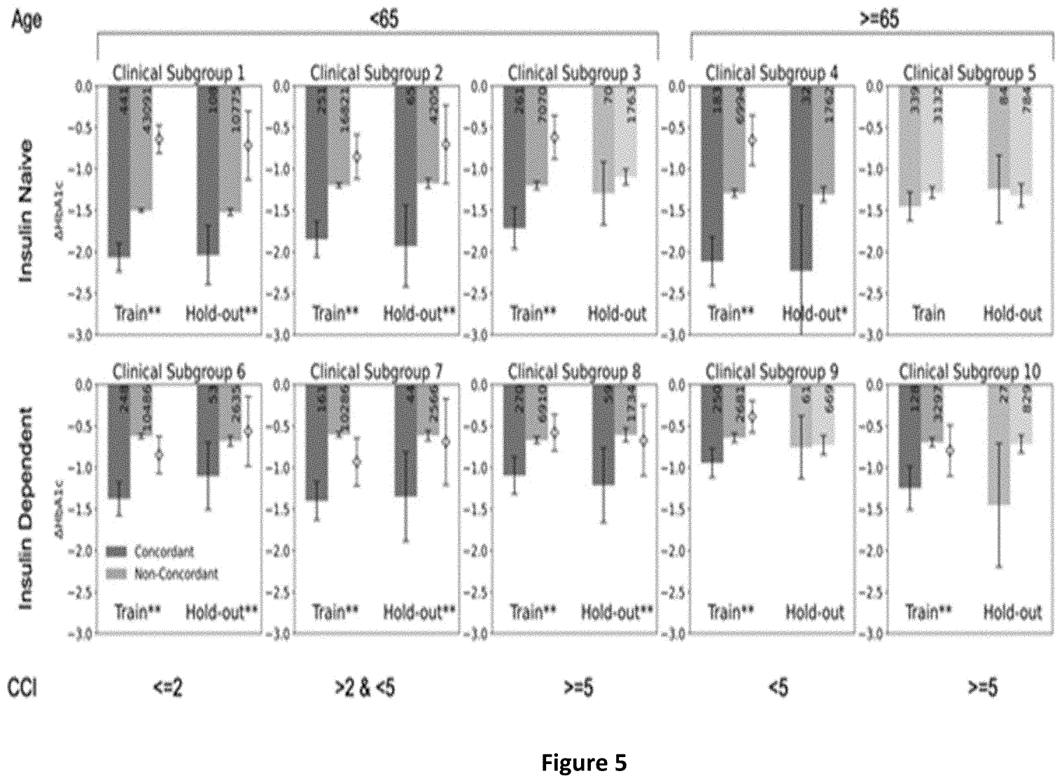

FIG. 5 shows change in lab measurements (HbA1c) for concordant (shown in blue) and non-concordant (shown in red) cohorts for all clinical subgroups, according to some implementations. An individual is considered concordant if their current treatment matches one of the top-K=3 recommendations for their clinical subgroup and non-concordant otherwise. Single asterisk in FIG. 5 denotes that the difference of means between concordant and non-concordant cohorts is statistically significant. A second asterisk denotes that the confounder-adjusted ATE of the causal recommender is also statistically significant. Diamonds show ATE values of the causal recommender for cases with two asterisks. Numbers on bars signify number of individuals in each cohort. CCI refers to Charlson Comorbidity Index. As observed from FIG. 5, concordant prescriptions result in a substantial reduction in HbA1C by the next lab measurement. This represents an improvement of approximately an additional 1% drop in A1C. Changes in A1C vs non-concordant (non-top-3 drug) are statistically significant even after adjusting for confounding between concordant and non-concordant patients. For example, in FIG. 5, the diamonds in the panels represent the confounder adjusted ATE values of the recommender systems. In some clinical subgroups, this value is ˜1%.

### Causal Recommender Sensitivity Analysis

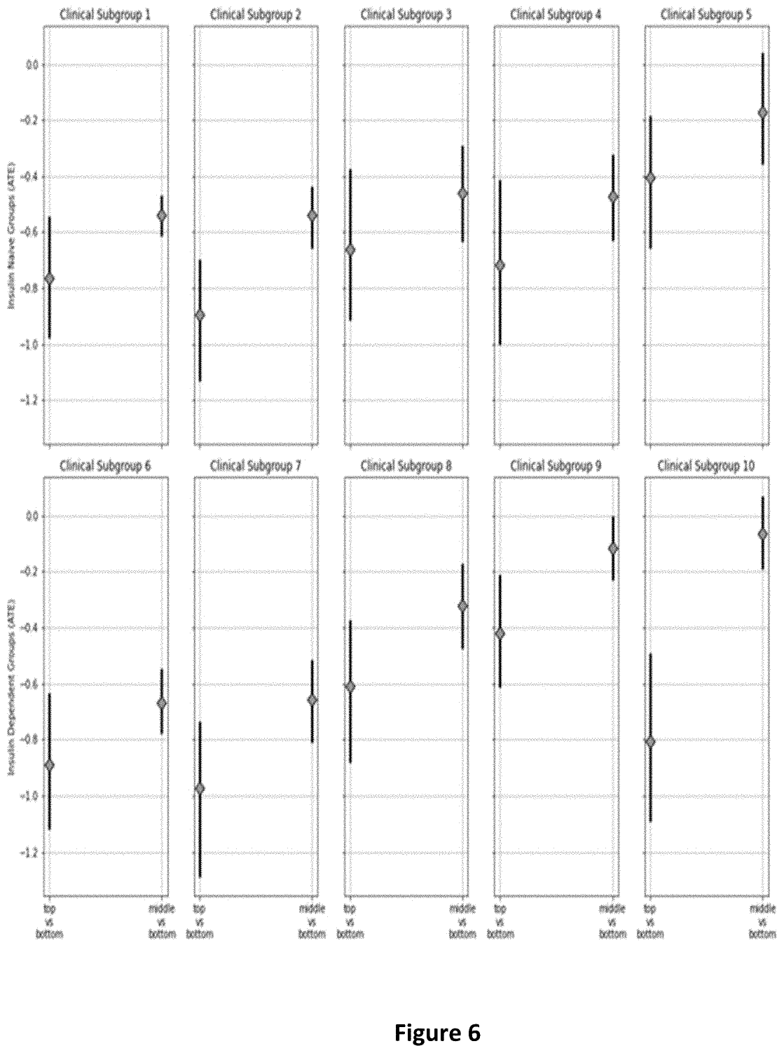

FIG. 6 depicts graph plots showing sensitivity analysis of recommender's ATE between 3 cohorts, according to some implementations. The “top” group represents patients who followed a recommendation in the top-3 rankings, “middle” group represents patients who followed a recommendation in the top 4-10 rankings and “bottom” group represents all other patients (who either followed a recommendation in ranking >10 or a treatment that was not ranked). A comparison of the efficacy of the recommender is shown for each clinical subgroup using 2 pairwise ATEs (first group representing the treatment group and second group representing the control group). In all cases, top-3 recommended treatments are shown to have significant positive effects (lowering HbA1c) when compared to a control cohort (“bottom” group). Recommendations in the 4-10 ranking positions are also seen to be beneficial, but of lower efficacy than top-3 recommendations. Note rank-related titration effects, the lower the treatment rank, the lower the resulting effect on A1C. Note also that although lowering A1C is the focus in the description here, some implementations instead optimize for distance from specified HbA1C targets.

The true measure of the impact of a recommender system for personalized medicine cannot be determined without conducting a well-controlled prospective study. However, the results of the retrospective study reported here may be indicative of what one might expect from a randomized experiment. A change in HbA1c in a diabetic population under managed care could lead to improvements in patient health outcomes that are substantive. By forestalling adverse events that arise from uncontrolled diabetes, it could reduce patient suffering and lead to significant reductions in healthcare costs.

The example causal recommender described here is optimized to reduce HbA1c. In some instances, absolute reductions in HbA1c may be not be desirable in certain sub-populations. For older patients, or for those with multiple comorbidities, for example, it may be more beneficial to reach appropriate targets set by the care manager. Some implementations generalize the causal recommender to optimize for meeting targets instead of absolute HbA1c reduction.

Treatment personalization in the causal recommender may be derived from two sources: (i) the stratification based on age, comorbidities, and prior insulin use and (ii) the censoring of contraindicated medications by looking through an individual's health history. The decision to use the stratification scheme described here is driven by clinical inputs. Some implementations use other stratification approaches that utilize machine learning algorithms, such as unsupervised k-means to discover natural clusters of similar patients or generating a bespoke recommender for each patient trained on the cohort of their k-nearest-neighbors. This latter approach provides a very high level of personalization but may come at the cost of increased computational overhead. Another layer of personalization is achieved by ranking treatments on Individual Treatment Effects (ITEs) instead of ATEs, according to some implementations.

The systems and methods described herein are readily extensible for making treatment recommendations for a variety of chronic diseases. In some implementations, training pipelines for causal recommenders is incorporated within the claims processing infrastructure of healthcare systems so that they can learn constantly and improve with time as more and newer data becomes available. For example, the causal recommender system describe herein could be incorporated within integrated EHR or claims processing systems so that online learning becomes possible (e.g., the model continues to learn and improve daily as more data comes in). In this way, causal recommenders can play an important role in personalizing medicine at the population level.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

What is claimed is:

1. A computer-implemented method for implementing a causal recommender for personalized disease treatment selection using machine learning, the method comprising:
obtaining health trajectories for patients, wherein each health trajectory corresponds to a respective patient and represents a time-ordered series of health events for the respective patient, wherein each health trajectory includes at least one health condition, at least one treatment event and at least one index event, wherein each health trajectory includes a respective plurality of sub-trajectories, and wherein each sub-trajectory includes a treatment event and ends at a respective index event;
stratifying the sub-trajectories for each patient to form a plurality of stratified patient segments, wherein each stratified patient segment corresponds to a separate and distinct health condition and includes the sub-trajectories for patients that have the health condition;
for each segment of the plurality of stratified patient segments:
performing pairwise causal inference analysis on one or more treatments corresponding to the sub-trajectories of the respective segment, to estimate average treatment effect (ATE) values;
performing network meta-analysis on the ATE values, thereby ranking the one or more treatments for each patient in the respective segment; and
in accordance with a determination that a respective patient has a health condition which could cause a set of treatments to be unsafe based on one or more clinical rules and the health trajectories, reranking the one or more treatments after excluding the set of treatments; and
outputting treatment options for personalized disease treatment selection for a patient based on ranked treatments for the plurality of stratified patient segments.

2. The method of claim 1, wherein performing the network meta-analysis includes:
constructing a densely connected network graph based on the ATE values; and
performing a Network Meta-Analysis (NMA) on the densely connected network graph.

3. The method of claim 2, wherein performing the Network Meta-Analysis (NMA) includes:
computing synthesized ATEs, for the ATE values, and computing the synthesized ATEs against a baseline treatment.

4. The method of claim 2, wherein performing the Network Meta-Analysis (NMA) includes:
computing a Surface Under the Cumulative RAnking curve (SUCRA) score for each treatment and ranking the one or more treatments according to the SUCRA curve.

5. The method of claim 1, wherein the pairwise causal inference analysis includes neural-network causal analysis to determine causal inference between each pair of treatments of the one or more treatments.

6. The method of claim 5, wherein the pairwise causal inference analysis estimates a total of N² unique ATE values per segment, where N is the number of treatments in the respective segment.

7. The method of claim 1, wherein the pairwise causal inference analysis uses inverse probability of treatment weighting (IPTW) method, where patients in control and treatment arms are assigned weights equal to the inverse probability for getting the treatment they received.

8. The method of claim 1, wherein stratifying the sub-trajectories comprises grouping patients on the basis of clinical covariates in the health trajectories.

9. The method of claim 1, wherein stratifying the sub-trajectories is performed by applying a machine learning algorithm on the health trajectories.

10. The method of claim 9, wherein the machine learning algorithm is an unsupervised k-means clustering that clusters similar patients based on treatments.

11. The method of claim 1, wherein stratifying the sub-trajectories is performed by generating a bespoke recommender for each patient trained on a cohort of their k-nearest-neighbors.

12. The method of claim 1, wherein stratifying the sub-trajectories includes splitting the health trajectories into segments based on age, prior treatment, and comorbidity index values.

13. The method of claim 1, further comprising:
selecting treatments that have at least a minimum cohort size to include in the one or more treatments.

14. The method of claim 1, further comprising:
for each patient of the plurality of patients:
identifying a respective treatment event and a respective index event in the health trajectory for the respective patient, wherein a respective index event is any clinical or health data point; and
segmenting the health trajectory into a respective plurality of sub-trajectories such that each sub-trajectory includes a treatment event and ends at a respective index event.

15. The method of claim 14, wherein each sub-trajectory terminates in a pair of lab measurements, the method further comprising:
computing age of the respective patient, any comorbidities, and prior medication as of the date of first lab of the pair of lab measurements in the sub-trajectory; and
using current medication as the treatment for the patient corresponding to the sub-trajectory, for the period between the two labs of the pair of lab measurements.

16. The method of claim 15, further comprising:
while segmenting the health trajectory, excluding sub-trajectories where the duration between the lab pairs is not within a predetermined time period.

17. The method of claim 15, further comprising:
removing patients with a single lab measurement from the plurality of patients, prior to segmenting the health trajectory.

18. The method of claim 15, further comprising:
when the respective patient has multiple medications, using a combination of the medications as the treatment for the respective patient for the period between the two labs of the pair of lab measurements.

19. The method of claim 1, further comprising:
for a new patient, recommending a personalized treatment option by identifying one or more sub-trajectories for a particular stratified patient segment that are most similar to the new patient's health trajectory.

20. A system for implementing a causal recommender for personalized disease treatment selection using machine learning, comprising:
one or more processors;
memory; and
one or more programs stored in the memory, wherein the one or more programs are configured for execution by the one or more processors and include instructions for:
obtaining health trajectories for patients, wherein each health trajectory corresponds to a respective patient and represents a time-ordered series of health events for the respective patient, wherein each health trajectory includes at least one health condition, at least one treatment event and at least one index event, wherein each health trajectory includes a respective plurality of sub-trajectories, and wherein each sub-trajectory includes a treatment event and ends at a respective index event;
stratifying the sub-trajectories for each patient to form a plurality of stratified patient segments, wherein each stratified patient segment corresponds to a separate and distinct health condition and includes the sub-trajectories for patients that have the health condition;
for each segment of the plurality of stratified patient segments:
performing pairwise causal inference analysis on one or more treatments corresponding to the sub-trajectories of the respective segment, to estimate average treatment effect (ATE) values;
performing network meta-analysis on the ATE values, thereby ranking the one or more treatments for each patient in the respective segment; and
in accordance with a determination that a respective patient has a health condition which could cause a set of treatments to be unsafe based on one or more clinical rules and the health trajectories, reranking the one or more treatments after excluding the set of treatments; and
outputting treatment options for personalized disease treatment selection for a patient based on ranked treatments for the plurality of stratified patient segments.
